(19) United States
(12) Patent Application Publication
(10) Pub. No.: US 2025/0366934 A1
(43) Pub. Date: Dec. 4, 2025

(54) SURGICAL ROBOTICS SYSTEMS AND DEVICES HAVING MODIFIED SPEED CONTROL NEAR WORKSPACE BOUNDARIES AND SURFACES, AND METHODS THEREOF

(71) Applicant: Distalmotion SA, Epalinges (CH)

(72) Inventors: Romain Michel MIRAN FARKAS, Cugy (CH); Patrick Schoeneich, Lausanne (CH); Walid Amanhoud, Anthy Sur Leman (FR); Laurent Renaud Uldry, Prilly (CH); Nicolas Paul Marcel Boch, Lausanne (CH)

(21) Appl. No.: 19/300,364

(22) Filed: Aug. 14, 2025

Related U.S. Application Data
(63) Continuation of application No. PCT/EP2024/054442, filed on Feb. 21, 2024.
(60) Provisional application No. 63/486,240, filed on Feb. 21, 2023.

(51) Int. Cl. A61B 34/20; A61B 34/30; A61B 34/37

(52) U.S. Cl. CPC A61B 34/20; A61B 34/37; A61B 2034/302

(57) ABSTRACT

The present disclosure relates to surgical robotic systems having a master console and slave manipulators, with components and features for adjusting movements of instruments near workspace boundaries. In some embodiments, a method of modulating movement of an articulated instrument arm and an instrument coupled thereto can include determining that the end effector is within a predefined zone adjacent to the boundary, and in response to determining that the end effector is within the predefined zone, controlling one or more actuators of an articulated instrument arm supporting the instrument to adjust a speed of the instrument.

### CROSS-REFERENCE TO RELATED APPLICATIONS

This application is a continuation of International Application No. PCT/EP2024/054442, filed on Feb. 21, 2024, which claims priority to U.S. Provisional Application No. 63/486,240, filed on Feb. 21, 2023, each of which is hereby incorporated by reference in its entirety.

### TECHNICAL FIELD

This application generally relates to surgical robot systems, and specifically to systems, devices, and methods for controlling and adjusting movements of instruments near workspace boundaries.

### BACKGROUND

In robotically-assisted or tele-manipulated surgical robotic systems, a surgeon operates a master console to remotely control one or more slave devices or surgical instruments at a surgical site. The surgical instruments are typically confined to movements within a workspace or spatial range, e.g., for safety or other operational reasons. During operation of the surgical instrument, however, there may be times when the tip of a surgical instrument is driven to a workspace boundary. In such instances, existing robotic systems may abruptly stop or modify the trajectory of the surgical instrument, i.e., to avoid breaching the boundary. An abrupt stop or modification of the instrument trajectory, however, may lead to an exaggerated counter-reaction from the surgeon, which can lead to further collateral damage or unexpected movements. As such, it may be desirable to have improved methods for controlling or modifying the motion of an instrument at a workspace boundary.

### BRIEF DESCRIPTION

The present disclosure overcomes the drawbacks of previously-known surgical robotic systems by providing systems, devices, and methods for adapting the motion of an instrument as the instrument and/or an articulated robotic arm is approaching or moving away from a workspace boundary.

In some embodiments, a system can include a human-machine interface for sensing user commands, a motorized and articulated robotic arm, and a surgical instrument with an end effector that is coupled to the robotic arm. A workspace may be defined by a set of predefined spatial boundaries, within which the tip of the surgical instrument and/or the articulated robotic arm is intended to move. In some embodiments, the workspace can be defined within a body cavity and/or externally of the body cavity (e.g., patient). Additionally or alternatively, one or more virtual fixtures can be defined during operation of the surgical robotic system. For example, the surgical robotic system can be configured to define one or more virtual fixtures in response to collisions and/or other conditions sensed or established during operation of the surgical robotic system. The system further includes a controller configured to process the user commands as inputs and to provide the commands to the articulated robotic arm as outputs. The controller can further adjust one or more movements of the articulated robotic arm to adjust the movement of the tip of the surgical instrument when the tip and/or the articulated robotic arm is near a workspace boundary.

In some embodiments, a method of modulating a translational movement of an end effector of an instrument near a boundary of a virtual workspace can include: determining that the end effector is within a predefined zone adjacent to the boundary; and in response to determining that the end effector is within the predefined zone, controlling one or more actuators of an articulated instrument arm supporting the instrument to move the instrument such that a direction of the translational movement of the end effector is maintained while a commanded speed of the translational movement of the end effector is modified based on a distance of the end effector to the boundary.

In some embodiments, a method of modulating a translational movement of an end effector of an instrument near a boundary of a virtual workspace can include: determining that the end effector is less than a predefined distance from the boundary; and in response to determining that the end effector is less than a predefined distance from the boundary, controlling one or more actuators of an articulated instrument arm supporting the instrument to move the instrument such that a direction of the translational movement of the end effector is maintained while a commanded speed of the translational movement of the end effector is modified based on a distance of the end effector to the boundary.

In some embodiments, an apparatus can include: an articulated instrument arm supporting an instrument including an end effector, the articulated instrument arm including one or more actuators that are configured to drive a movement of the instrument within a virtual workspace defined by a boundary within a body cavity; and a controller operatively coupled to the articulated instrument, the controller configured to: monitor a position of the end effector within the body cavity; determine, based on monitoring the position of the end effector, that the end effector is within a predefined zone adjacent to the boundary; and in response to determining that the end effector is within the predefined zone, controlling the one or more actuators to move the instrument such that a direction of the translational movement of the end effector is maintained while a commanded speed of the translational movement of the end effector is modified based on a distance of the end effector to the boundary.

In some embodiments, a method of modulating movement of an articulated instrument arm and an instrument coupled thereto includes: determining a position of a distal portion of the articulated instrument arm relative to a virtual surface disposed outside of a body of a patient; determining a position of an end effector of the instrument relative to a boundary of a virtual workspace disposed within the body of the patient; adjusting a commanded speed vector of the articulated instrument arm based on at least on one of a distance between the position of the distal portion of the articulated instrument arm and the virtual surface or a distance between the position of the end effector and the boundary of the virtual workspace, to produce a modified speed vector; and controlling one or more actuators of the articulated instrument arm to move the articulated instrument arm and the instrument based on the modified speed vector.

In some embodiments, a method of modulating movement of an articulated instrument arm and an instrument coupled thereto includes: determining whether an end effector of the instrument is within a predefined zone adjacent to a boundary of a virtual workspace; and determining whether a distal portion of the articulated instrument arm is less than a predefined distance from a virtual surface; in response to determining that the end effector is within the predefined zone and the distal portion of the articulated instrument arm is less than the predefined distance from the virtual surface, adjusting a commanded speed of the articulated instrument arm to adjust a commanded speed of translational movement of the end effector while preventing the articulated instrument arm from intersecting the virtual surface.

In some embodiments, a method of modulating movement of an articulated instrument arm and an instrument coupled thereto includes: monitoring a position of the articulated instrument arm outside a body cavity while an end effector of the instrument is disposed within a virtual workspace of the body cavity; determining, based on monitoring the position of the articulated instrument arm, that a distal portion of the articulated instrument arm is less than a predefined distance from a virtual surface; and in response to determining that the distal portion of the articulated instrument arm is less than the predefined distance from the virtual surface, controlling one or more actuators of the articulated instrument arm to adjust a commanded speed of the distal portion of the articulated instrument arm while maintaining the end effector within the virtual workspace.

### BRIEF DESCRIPTION OF THE DRAWINGS

FIG. 1 schematically depicts a surgical robotic system, according to embodiments.

FIG. 2 schematically depicts a surgical robotic system with a controller configured to adapt movements of an instrument near a workspace boundary, according to embodiments.

FIG. 3 schematically depicts movements of an instrument of a surgical robotic system near a workspace boundary, according to embodiments.

FIG. 4A schematically depicts movements of an instrument of a surgical robotic system near a workspace boundary with symmetric movement control, according to embodiments.

FIG. 4B schematically depicts movements of an instrument of a surgical robotic system near a workspace boundary with asymmetric movement control, according to embodiments.

FIG. 5A schematically depicts a three-dimensional view of a workspace for an instrument of a surgical robotic system, according to embodiments.

FIG. 5B depicts a cross-sectional view of the workspace for an instrument of a surgical robotic system and changes applied to a commanded speed of the instrument as a function of instrument position relative to a boundary of the workspace, according to embodiments.

FIG. 6 schematically depicts a graph showing a gain or modulation factor that can be used to adjust the commanded speed of a slave manipulator of a surgical robotic system, based on a normal distance of the slave manipulator to a virtual fixture and normal commanded speed, according to embodiments.

FIGS. 7, 8, and 9 are flow charts of example methods for controlling movements of an instrument of a surgical robotic system, according to embodiments

FIG. 10 schematically depicts the motion control of a slave manipulator of a surgical robotic system, according to embodiments.

### DETAILED DESCRIPTION

The present disclosure relates to surgical robotic systems having a master console and one or more slave manipulators, with components and features for adapting the motion of an instrument coupled to a slave manipulator. Systems, devices, and methods described herein can reduce movements of an instrument and/or the articulated robotic arm of a surgical robotic system as the instrument and/or the articulated robotic arm are approaching or moving away from a respective boundary of a workspace and virtual object (e.g., virtual boundary).

During a surgical operation, a surgical robotic system may be used to perform certain minimally invasive procedures. In some embodiments, the surgical robotic system may include one or more patient-side carts and a surgeon or master console. The patient-side carts may include robotic arms that support one or more instruments, which may be used during the surgical operation. During a surgical procedure, a surgeon or other operator may provide inputs at the master console, which are translated to movements of the robotic arms and the instruments that are supported thereon. Generally, the Instruments are confined to movements within a predefined workspace, e.g., for safety or other operational reasons. As such, when a tip of an instrument reaches a boundary of a workspace, the robotic arm supporting the instrument may stop further movement of the instrument and/or modify its trajectory. Similarly, the surgical robotic system can be configured to avoid undesirable movement of the robotic arm (e.g., into an unsafe space) or to prevent collision with physical objects in the real world. For example, the surgical robotic system can be configured to define virtual fixtures (e.g., objects, boundaries, surfaces, etc.) that can be associated with unsafe spaces or physical objects in the real world (e.g., patient-side cart, master console, wall, patient platform), and in controlling the robotic arm, the surgical robotic system can be configured to prevent intersection of the arm with these virtual fixtures. More specifically, when a portion of a robotic arm is within a predetermined distance from a virtual object (e.g., a predefined normal distance to a surface), the system may modify the movement (e.g., commanded speed, direction, trajectory) of the robotic arm to avoid an intersection of the arm and the virtual object. While described with respect to the robotic arm, it can be appreciated that virtual fixtures can be define for other portions of the surgical robotic system, including, for example, the master manipulator, the slave console or cart, etc.

When a robotic system abruptly stops the movement of an instrument and/or modifies its trajectory, the abrupt change can result in vibrations or shaking of the mechanical structure of the robotic arm, which can produce distortion of the instrument tip movement. Such movements may not be expected by the surgeon or operator and may therefore lead to an exaggerated counter-reaction from the surgeon or operator. This can lead to further unexpected movements and/or collateral damage. Therefore, it can be desirable to have systems and methods that are configured to modulate or change the commanded speed or movement of a robotic arm (or other part of a surgical robotic system) as a function of the relative position of the robotic arm or instrument to a virtual boundary, fixture, or surface, the direction and/or magnitude of the commanded speed of their movement, and/or other parameters. The speed modulation can be implemented progressively, e.g., as the robotic arm and/or instrument approaches or gets closer to a virtual boundary, fixture, or surface, or gets away from a virtual boundary, fixture, or surface.

In some cases, a surgical robotic system can include user feedback devices (e.g., visual, audio, etc.) that provide feedback to a user when an instrument and/or robotic arm being controlled by the user is nearing a respective workspace boundary and virtual object or surface. For example, the system can send the user a signal as the instrument is approaching a boundary and/or the robotic arm is approaching a virtual object or surface so that the user knows to adapt his commands to avoid hitting or breaching the boundary with the instrument and/or the virtual object with the robotic arm. If, however, a user does not adapt his commands in response to the signal, then the system may still generate unexpected movements when the instrument hits the boundary and/or the robotic arm hits the virtual object or surface.

Systems, devices, and methods described herein can avoid these issues by reducing or minimizing unexpected movements of instruments near workspace boundaries and/or unexpected movements of robotic arms near virtual objects, e.g., by adapting robotic arm motion as an instrument approaches or moves away from a virtual workspace boundary, or by adapting robotic arm motion as a portion of the robotic arm approaches or moves away from a virtual fixture.

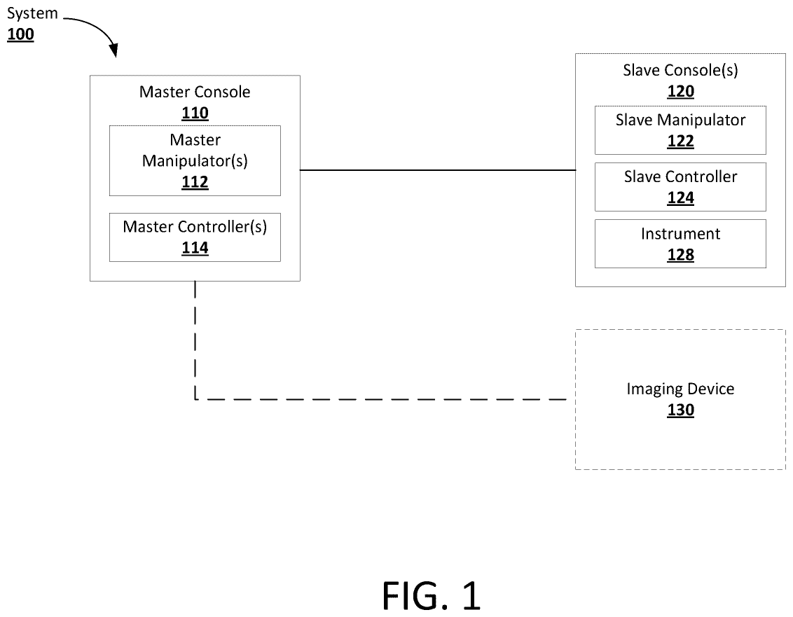

FIG. 1 schematically depicts a surgical robotic system 100, according to embodiments. The system 100 can include a master console 110 and one or more slave console(s) 120. Optionally, the system 100 can also include an imaging device 130, such as, for example, an endoscopic camera.

The master console 110 can be operatively coupled to the slave console(s) 120. For example, the master console 110 can be coupled to the slave console(s) 120 via wired and/or wireless connections. The master console 110 can include one or more master manipulator(s) 112 and one or more master controller(s) 114. The master manipulator(s) 112 can include a plurality of master links that are interconnected by a plurality of joints. Movement can be applied to the master manipulator(s) 112 by a handle (for instance a sterile handle), which can be actuated by a user (for instance a sterile user, e.g., a surgeon). The movement of the master manipulator(s) 112 and one or more actuators of the handle can be sensed, e.g., using a plurality of sensors, and transmitted to the master controller(s) 114. In operation, the master controller(s) 114 can send instructions to one or more slave console(s) 120 to cause one or more drive units and/or actuators at the slave console(s) 120 to move based on the movements applied at the master console 110.

Each slave console 120 can include a slave manipulator 122 and/or an instrument 128 that is coupled to the slave manipulator 122. The slave manipulator 122 can be implemented as a robotic arm, e.g., including a plurality of links that are interconnected by a plurality of corresponding joints. The slave console(s) 120 can include one or more drive units and/or actuators that control movement of the plurality of links and joints of the slave manipulator 122. The instrument 128 can be removably coupled to the slave manipulator 122. When the instrument 128 is coupled to the slave manipulator 122, the slave manipulator 122 can be configured to support the instrument 128 and to control its movements. In particular, the slave manipulator 122 can be configured to control and move the instrument 128 in a plurality of degrees of freedom (DOF), including translational and/or rotational movement. The slave manipulator 122 can be configured to control the movements of the instrument 128 in a manner responsive to movements applied at the handle of the master console 110. In particular, the master console 110 can generate instructions or commands based on movements applied at the handle and transmit those instructions or commands to the slave console(s) 120 to cause movement of the slave manipulator 122 and/or the instrument 128. The slave console(s) 120 can include a slave controller 124 that can be configured to interpret the instructions or other signals from the master console 110 and to control the movement of the slave manipulator 122 and/or the instrument 128.

While the slave console 120 is described as having a slave manipulator 122 and an instrument 128, it can be appreciated that a single slave console 120 can include more than one slave manipulator 122 and/or more than one instrument 128. For example, a slave console 120 can include two slave manipulators 122 that each support one or more instruments 128.

The master controller(s) 114 and the slave controller(s) 124, as described herein, can include one or more of a memory, a processor, a communications interface, and/or an input/output device. The memory can include any type of suitable non-transitory computer readable media that can store instructions that can be executed by one or more processors. The memory can be, for example, a random access memory (RAM), a memory buffer, a hard drive, a database, an erasable programmable read-only memory (EPROM), an electrically erasable read-only memory (EEPROM), a read-only memory (ROM), and/or so forth. The processor can be any suitable processing device configured to run and/or execute functions associated with the surgical robotic system 100. The processor can be a general purpose processor, a Field Programmable Gate Array (FPGA), an Application Specific Integrated Circuit (ASIC), a Digital Signal Processor (DSP), and/or the like. The communications interface can include wired and/or wireless interfaces for receiving information and/or sending information to other devices. The input/output device can include one or more displays, audio devices, touchscreens, keyboards, or other input or output devices for presenting information to and/or receiving information from a user.

Further examples of surgical robotic systems are described in PCT Patent Application No. PCT/IB2020/050039, filed Jan. 4, 2020, titled “Surgical Robot Systems Comprising Robotic Telemanipulators and Integrated Laparoscopy,” and U.S. patent application Ser. No. 16/269,383, filed Feb. 6, 2019, titled “Surgical Robot Systems Comprising Robotic Telemanipulators and Integrated Laparoscopy,” the disclosures of each of which are incorporated by reference herein.

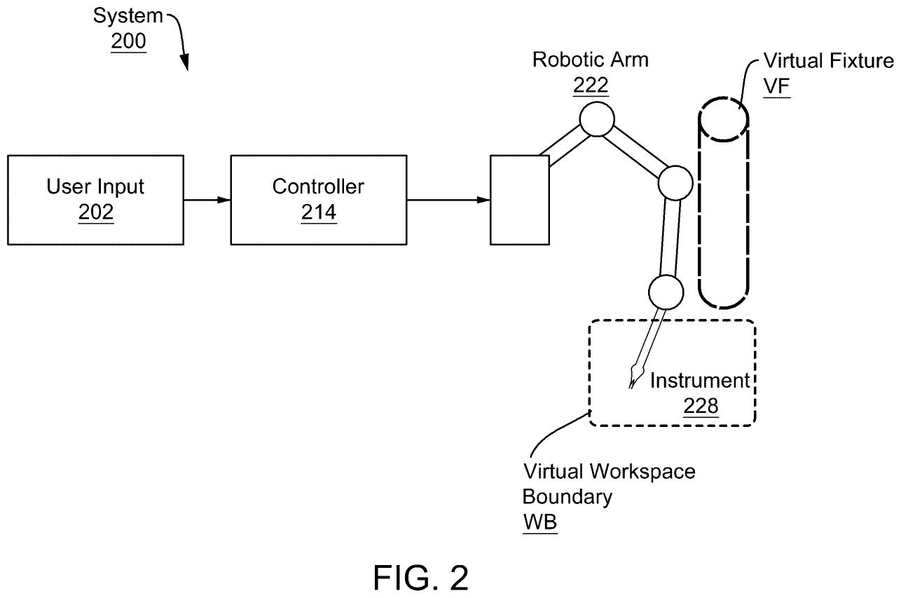

FIG. 2 schematically depicts a surgical robotic system 200 including a controller 214 that is configured to modulate or modify movements of an instrument 228, according to embodiments. The surgical robotic system 200 can be structurally and/or functionally similar to other surgical robotic systems described herein (e.g., the surgical robotic system 100), and therefore can include components that are structurally and/or functionally similar to those of other surgical robotic systems described herein. For example, the controller 214 can be structurally and/or functionally similar to the master controller 114 and/or slave controller 124, the robotic arm 222 can be structurally and/or functionally similar to the slave manipulator 122, and the instrument 228 can be structurally and/or functionally similar to the instrument 128.

As depicted in FIG. 2, one or more user input(s) or command(s) 202 can be received at the controller 214. The controller 214 can include or be operatively coupled to a human-machine interface configured to sense the user input(s) 202. For example, the controller 214 can be operatively coupled to one or more master manipulators (e.g., master manipulators 112), and be configured to sense movement of those manipulators to determine a user input 202. The controller 214 can be operatively coupled to the robotic arm 222, which can be a motorized and articulated robotic arm (e.g., articulated instrument arm). The robotic arm 222 can support the instrument 228, which can be a surgical instrument having an end effector. In embodiments, the robotic arm 222 can include a hub or a distal end that can be configured to receive and couple to the instrument 228. Once the instrument 228 is coupled to the hub of the robotic arm 222, the instrument 228 can be confined to movements within a workspace defined by a set of predefined spatial boundaries or virtual boundaries WB. In particular, the end effector or tip of the instrument 228 can be limited to movements within the workspace. In embodiments, the workspace can be defined within a body of a patient (e.g., with a lumen or cavity of a patient), or the workspace can be defined outside the body of a patient (e.g., near a surgical site or patient anatomy of interest).

Additionally or alternatively, the robotic arm 222, instrument 228, or other portion of the system 200 can be configured to be non-intersecting with one or more virtual fixtures VF. These virtual fixtures VF can be predefined fixtures that are defined outside of the body of a patient (e.g., external to a body cavity) and/or further away from a surgical site. The virtual fixtures VF can be, for example, a surface, wall, line, two-dimensional shape (e.g., triangle, square, etc.), three-dimensional shape (e.g., sphere, cylinder, cone), or other more complex shape. The virtual fixtures VF can be associated with one or more objects in the real world (e.g., furniture, surgical devices, robotic devices, patient bed, carts, etc.) or zones that the robotic arm 222, instrument 228, or other portion of the system 200 should avoid (e.g., unsafe zones, zones that may obstruct the movements or sight of a surgeon and/or other healthcare practitioner, zones that need to remain clear for allowing other interventions, etc.). In some embodiments, the virtual fixtures VF can be defined in response to a previous collision with an object. The virtual fixture VF can act similarly to the workspace boundary WB by modulating (e.g., reducing) the robot In embodiments, the virtual boundary WB and the virtual fixtures VF can be defined separately from one another. Alternatively, the virtual boundary WB and virtual fixtures VF can be defined in relation with or together with one another.

The controller 214 can include a memory and a processor, e.g., similar to the controller 114 and/or the controller 124 described above. The memory can store instructions (e.g., software) that, when executed or implemented by the processor, causes the processor to process one or more user commands as inputs 202 and to generate signals or commands to one or more motors of the robotic arm 222 based on the inputs 202. In embodiments described herein, the controller 214 can command one or more motors of the robotic arm 222 to move the instrument 228 such that a direction of the movement of the end effector of the instrument 228 is maintained while a commanded speed of the movement of the end effector of the instrument 228 is modified when the end effector is within a predefined distance of a workspace boundary WB. In embodiments described herein, the controller 214 can command one or more motors of the robotic arm 222 to move such that a direction of the movement of the instrument 228 and/or the robotic arm 222 (or portion(s) thereof) is maintained while a commanded speed of the movement of the instrument 228 and/or the robotic arm 222 (or portion(s) thereof) is modified when the robotic arm 222 (or portion thereof) is within a predefined distance of a virtual fixture VF.

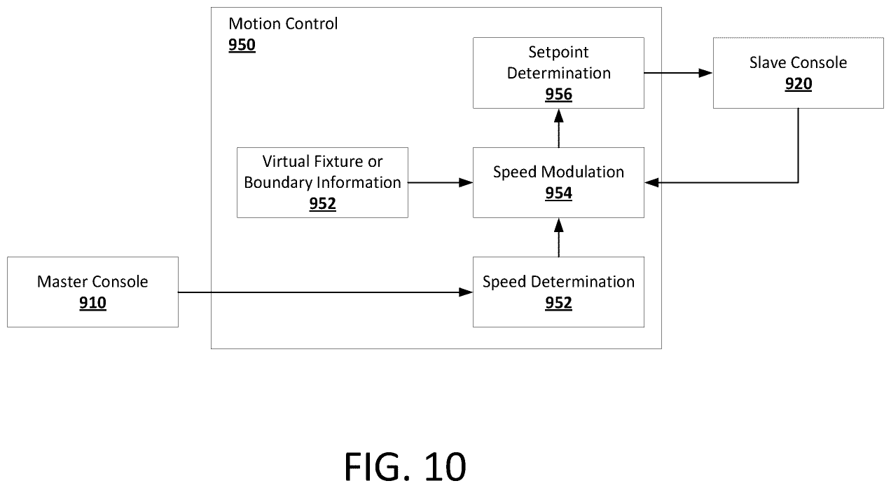

In embodiments, the controller 214 can be configured to control the movement of the robotic arm and the instrument coupled thereto based on a speed vector commanded by a surgeon through a master console (e.g., master console 110). The robotic arm can be driven in movement by a set of motors. The controller 214 can be configured to determine motor setpoints for the set of motors based on the speed vector. In some embodiments, the controller 214 can be configured to adapt or modify the speed vector commanded by the surgeon based on the distance of the end effector to a workspace boundary WB, whether the end effector is moving toward or away from a workspace boundary WB, the magnitude of the commanded speed of movement of the end effector, the distance of the robotic arm 222 or portion thereof (e.g., a hub of the robotic arm) to a virtual fixture VF, whether the robotic arm 222 is moving toward or away from a virtual fixture VF, the magnitude of the commanded speed of movement of the robotic arm 222, and/or other parameters. In some embodiments, one or more functions defined based on these parameters can be used to modulate the speed vector, e.g., by applying the function(s) to calculate a new or modified speed vector. Further details of this are described with reference to FIG. 10 below. In embodiments, the controller 214 can be configured to determine and monitor the distance of a distal end or hub of the robotic arm 222 relative to a virtual fixture VF and/or the distance of the end effector of the instrument 228 relative to a workspace boundary WB based on a kinematic model. In embodiments, the controller 214 can be operatively coupled to one or more sensors disposed on the robotic arm 222 and/or instrument 228 and determine the distance of a distal end or hub of the robotic arm 222 relative to a virtual fixture VF and/or the distance of the end effector of the instrument 228 relative to a workspace boundary WB based on signals received from the sensors. For example, physical markers, position sensors, encoders, and/or other mechanisms can be used to track the location of the hub of the robotic arm 222 and/or end effector of the instrument 228.

In embodiments, the modification of the commanded speed of the robotic arm 222 and/or instrument 228 can vary, e.g., depending on the distance of the end effector to the workspace boundary WB and/or whether the end effector is moving toward or away from the workspace boundary WB. In embodiments, the modification of the commanded speed of the robotic arm 222 and/or instrument 228 can vary, e.g., depending on the distance of the end effector to the workspace boundary WB, the commanded speed and direction of movement of the end effector toward or away from the workspace boundary WB, the distance of the robotic arm 222 (or hub) to a virtual fixture VF, and/or the commanded speed and direction of movement of the robotic arm 222 (or hub) toward or away from a virtual fixture VF.

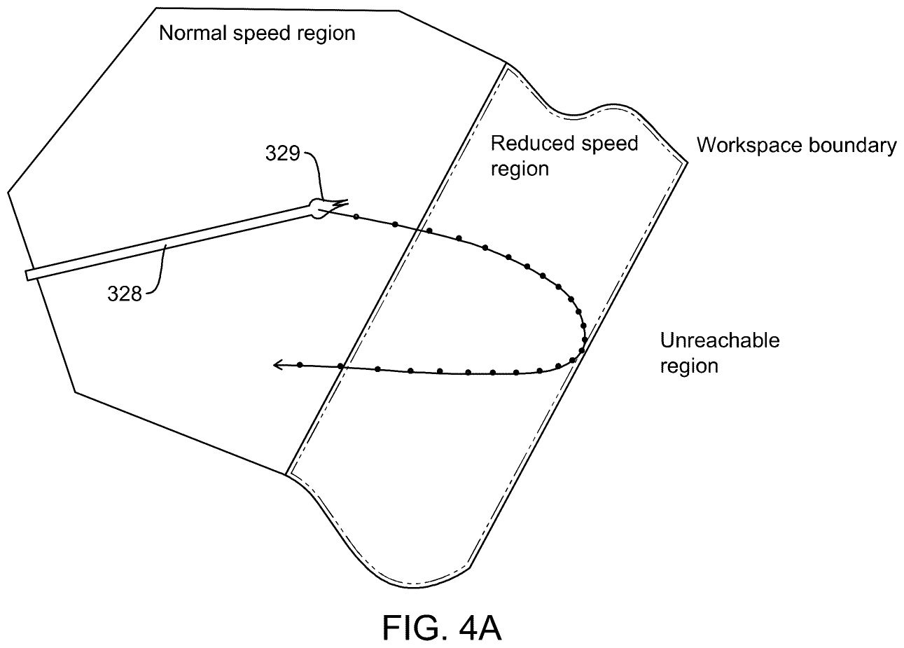

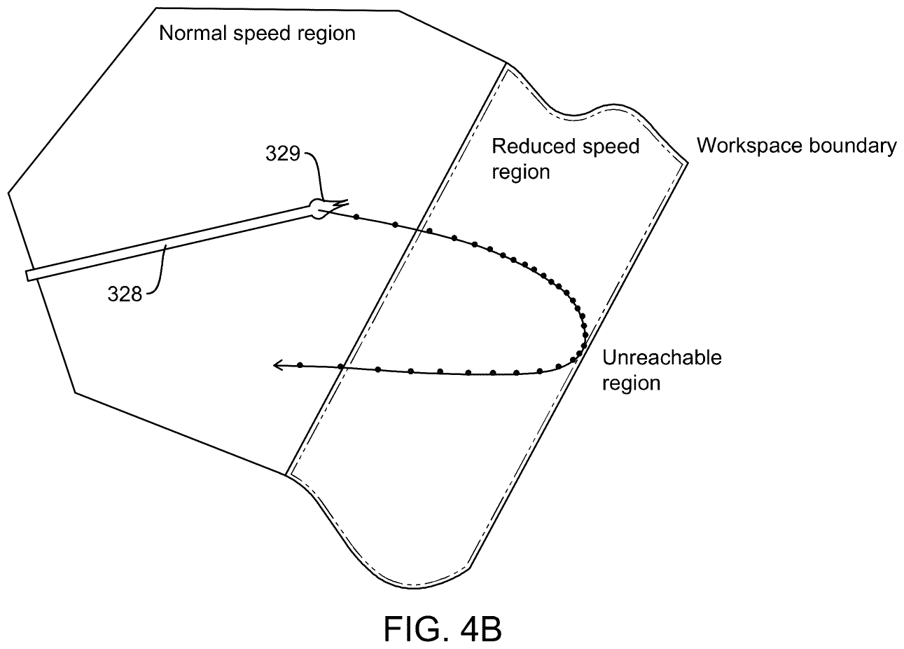

In embodiments, the controller 214 can be configured to modify the translational movement of the end effector by a percentage (e.g., between 0% and 100%, including all sub-ranges and values therebetween) or factor (e.g., between 0 and 1, including all sub-ranges and values therebetween), where the percentage or factor can be a function of the distance of the end effector to the workspace boundary WB. The function can define a direct relationship between the percentage or factor and the distance of the end effector to the workspace boundary WB. In other words, the percentage or factor as determined by the function can decrease as the distance of the end effector to the boundary decreases and can increase as the distance of the end effector to the boundary increases. Such a modification can be a symmetric modification of the commanded speed of the end effector. Alternatively, in some embodiments, the controller 214 can be configured to modify a commanded speed of the translational movement of the end effector by a percentage or factor, where the percentage or factor can be determined based on the distance of the end effector to the workspace boundary WB and whether the end effector is getting closer to or further from the workspace boundary WB. For example, the commanded translational speed of the end effector can be modified by a percentage or factor as determined by a first function when the direction of the translational movement is toward the workspace boundary WB and by a percentage or factor as determined by a second function that is different from the first function when the direction of the translational movement is away from the workspace boundary WB. In some embodiments, the percentage or factor as determined by the first function can decrease at a first rate as the distance of the end effector to the boundary WB decreases, and the percentage as determined by the second function can increase at a second rate that is different from the first rate (e.g., greater than or less than the first rate) as the distance of the end effector to the boundary WB increases. Such a modification can be an asymmetric modification of the commanded speed of the end effector. Further details of such modifications are described below with reference to FIGS. 4A and 4B.

In some embodiments, the controller 214 can be configured to modify the movement of the robotic arm by using a modulation gain or other factor to modulate the speed vector commanded by the surgeon via the master console. For example, a modulation gain, a_{VF}, can be defined as a function of the speed vector commanded by a surgeon, \dot{x}_{N}∈ [symbol] , and a position of the hub of the robotic device in Cartesian space or relative to a virtual fixture, x∈ [symbol] . The controller 214 can be configured to modulate the speed vector as follows:

$${\dot{x}}_{M} = {a}_{V F} ( {\dot{x}}_{N} , x ) * {\dot{x}}_{N}$$

where a_{VF}(\dot{x}_{N},x)∈[0,1] is a modulation gain that attenuates the commanded speed vector if the vector is moving the hub of the robotic arm closer to a virtual fixture VF, i.e., when a_{VF}(\dot{x}_{N},x)→0. As noted above, the virtual fixtures can take the form of a wall, surface, sphere, cylinder, or other more complex shape or geometry. The controller 214 can be configured to define the virtual fixtures VF, and then use position or distance and commanded speed to the virtual fixture VF to construct a_{VF}(\dot{x}_{N},x).

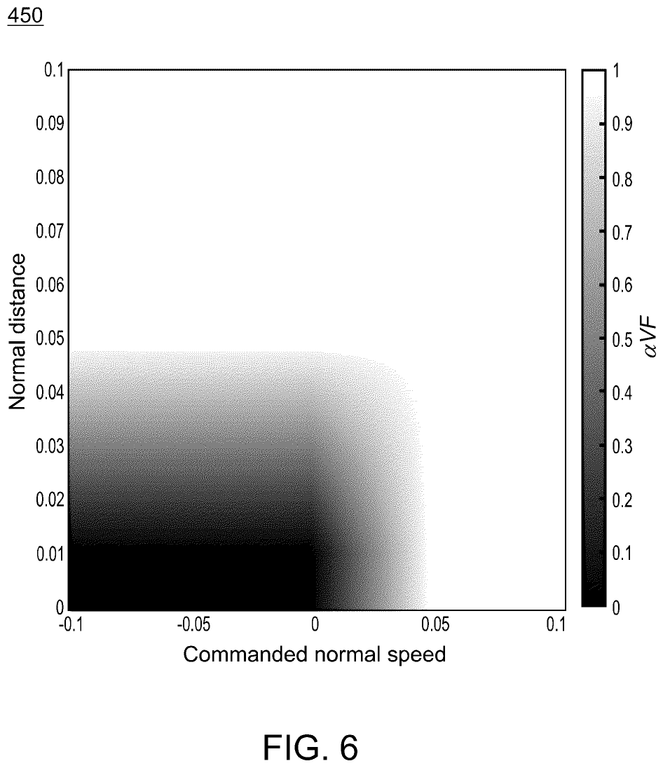

In embodiments where the virtual fixture includes a surface or wall, the gain can be defined as a function of the commanded normal speed and the normal distance of the hub to the surface of the virtual fixture. FIG. 6 illustrates a plot 450 of gain, a_{VF}, as a function of the normal distance and the commanded normal speed to the surface of a virtual fixture VF. The commanded normal speed is defined as being positive when the surgeon is commanding movement away from the virtual fixture VF and negative when the surgeon is commanding movement toward the virtual fixture VF. As depicted, the gain tends to zero when the normal distance decreases and the commanded normal speed is negative, and the gain converges to one when the normal distance increases and/or the commanded normal speed is positive.

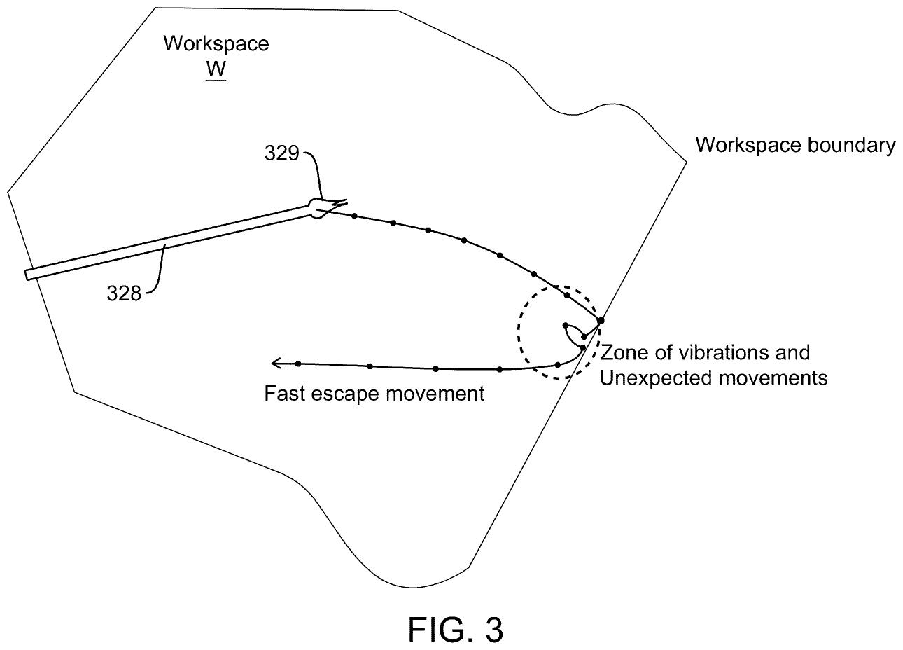

FIG. 3 schematically depicts the movements of an end effector or tip 329 of an instrument 328 of a surgical robotic system, where no adjustments are made to a commanded speed of the end effector 329 prior to reaching the workspace boundary. As depicted, when the end effector 329 hits or reaches the workspace boundary, there can be vibrations or other unexpected movements of the end effector 329 as the end effector 329 is stopped or slowed, e.g., to prevent the end effector 329 from breaching or extending further beyond the workspace boundary. In some cases, a surgeon may over-react to hitting the workspace boundary and command the instrument 328 to perform a fast escape movement. The vibrations, fast escape movement, and/or other unexpected movements may increase the risk of collateral damage.

FIGS. 4A and 4B schematically depict the movements of the end effector 329 of the instrument 328, where adjustments are made to a commanded speed of the movements of the end effector 329, according to embodiments. FIG. 4A depicts the movements of the end effector 329 where the commanded speed of the end effector 329 is adjusted symmetrically based on the distance of the end effector to the workspace boundary. As shown, when the distance of the end effector 329 to the workspace boundary decreases, the speed of the end effector decreases, e.g., according to a function. And when the distance of the end effector 329 to the workspace boundary increases, the speed of the end effector increases, e.g., according to the same function. FIG. 4B depicts the movements of the end effector 329 where the commanded speed of the end effector 329 is adjusted asymmetrically based on whether the end effector 329 is approaching or moving away from the workspace boundary. As shown, when the end effector 329 is approaching the boundary and the distance of the end effector 329 to the boundary is decreasing, the speed of the end effector 329 decreases, e.g., according to a function. And when the end effector 329 is moving away from the boundary and the distance of the end effector 329 to the boundary is increasing, the speed of the end effector 329 increases but in a different manner than when the end effector 329 is moving toward the boundary, e.g., according to a different function. In other words, in the asymmetric case, depending on whether the end effector 329 is moving toward or away from the boundary, the commanded speed of the end effector 329 may be adjusted differently, e.g., according to first and second functions that are different from one another. In some embodiments, the asymmetric case may allow a surgeon to move the instrument 328 more quickly away from a workspace boundary. For example, when the end effector 329 is moving away from the workspace boundary, the speed of the end effector 329 in the asymmetric case may increase faster than that of the symmetric case. Alternatively, the asymmetric case may allow a surgeon to move the instrument 328 more slowly away from a workspace boundary. In other words, the percentage or factor as determined by the first function decreases at a first rate as the distance of the end effector to the boundary decreases, and the percentage or factor as determined by the second function increases at a second rate as the distance of the end effector to the boundary increases, the second rate being inferior to the first rate.

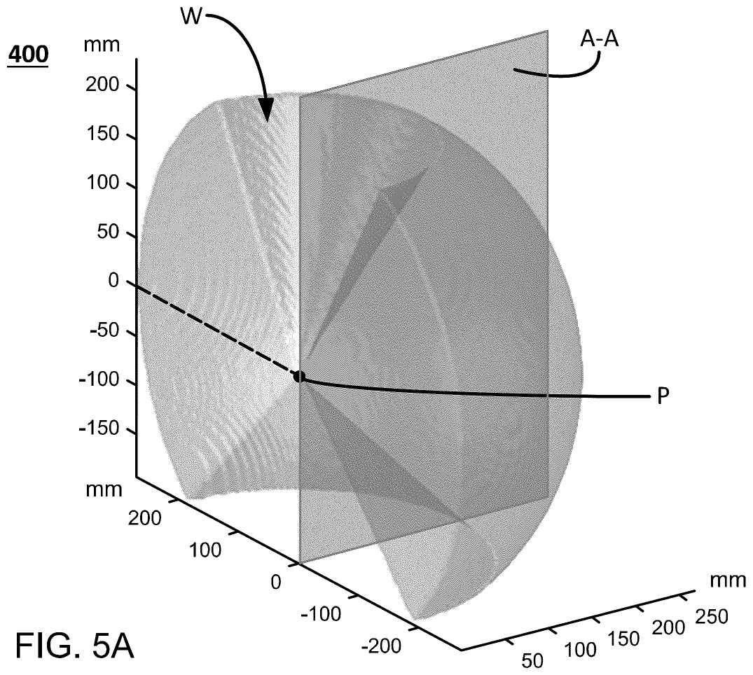

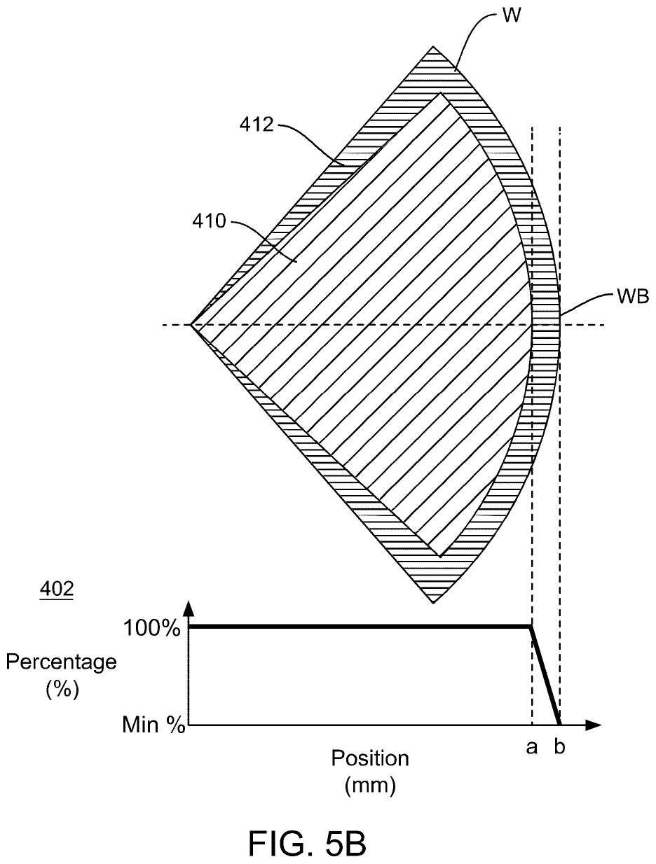

FIG. 5A depicts a plot 400 of an example workspace W of an instrument of a surgical robotic system, according to embodiments. The workspace W can be a workspace defined around an insertion point P into a body of a patient. As show, the workspace W can have a conical shape that increases in radius with increasing depth or distance into the body. FIG. 5B depicts a cross-sectional view of the workspace W, taken along a plane A-A of FIG. 5A, along with a plot 402 of a percentage or adjustment being applied to the commanded speed of the instrument tip or end effector as a function of the position of the end effector relative to a boundary WB of the workspace W. The commanded speed of the instrument tip can be unadjusted when the instrument tip position is in a first zone 410 that is sufficiently far away from the boundaries of the workspace W, while the commanded speed of the instrument tip can be adjusted when the instrument tip position is in a second zone 412 that is near at least one boundary of the workspace W. In some embodiments, the second zone 412 can be a predefined zone that is defined based on a set distance away from a workspace boundary or a set angle of the instrument relative to a workspace boundary (e.g., an angle between an instrument shaft and a planar boundary, or an angle of the line extending from the point of instrument insertion to the instrument tip and a planar boundary).

As shown in plot 402, the commanded speed of the instrument tip or end effector can be adjusted according to a decreasing function when the instrument tip is in the second zone 412, i.e., between positions a and b in the plot 402. While a linear decreasing function is shown in FIG. 5B, it can be appreciated that any type of decreasing function (e.g., exponential, logarithmic, etc., or combination thereof) can be used to modify or adapt the commanded speed of the end effector. The speed of the end effector can be decreased to a predefined minimum percentage or factor, e.g., about 1%, about 5%, about 10% or about 15% of a commanded speed of the end effector, including all ranges and values therebetween.

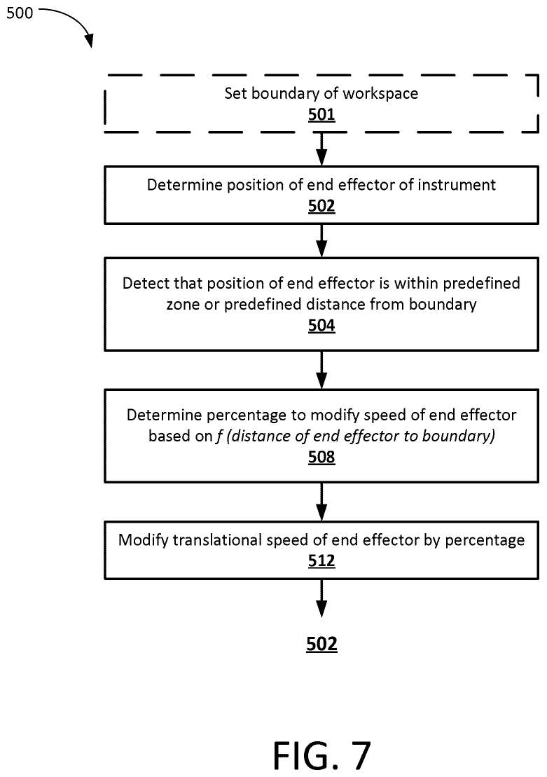

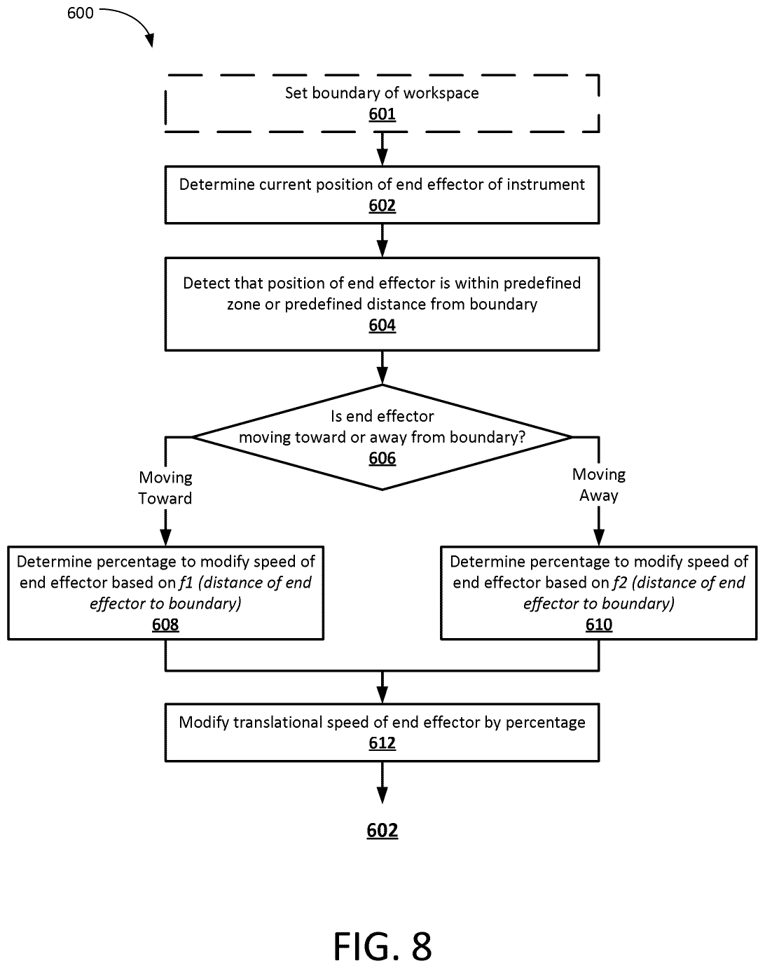

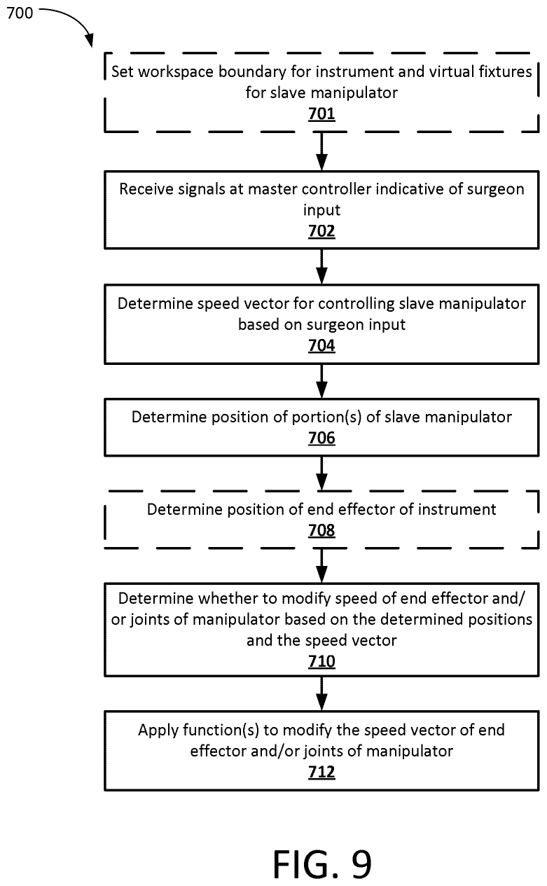

FIGS. 7-9 depict flow charts of example methods for controlling movements of an instrument and/or slave manipulator (e.g., robotic arm) of a surgical robotic system, according to embodiments. FIG. 7 depicts a method 500 that implements a symmetric adjustment to a commanded speed of a tip or end effector of the instrument based on a workspace boundary. FIG. 8 depicts a method 600 that implements an asymmetric adjustment to a commanded speed of a tip or end effector of the instrument based on a workspace boundary. FIG. 9 depicts a method 700 that implements an adjustment to the commanded speed of a slave manipulator (e.g., robotic arm) and the end effector of an instrument coupled thereto based on a workspace boundary and one or more virtual fixtures. The methods 500, 600, and 700 can be implemented by any of the surgical robotic systems described herein, including, for example, surgical robotic systems 100 and 200. In particular, the methods 500, 600, and 700 can be implemented by one or more processors and/or controllers (e.g., master controller(s) 114, slave controller(s) 124, controller 214) of the surgical robotic systems described herein.

The method 500 can include optionally defining or setting one or more boundaries of a workspace, at 501. In some embodiments, the virtual workspace is defined by a boundary within a body cavity. For example, a surgeon or other operator via a controller (e.g., master controller(s) 114, slave controller(s) 124, controller 214) may set one or more boundaries of a workspace based on parameters, including, for example, the dimensions of the body lumen in which the instrument is operating, the type and/or dimensions of the instrument, etc. These boundaries can be set before or after the surgeon or operator positions an instrument within a target body lumen. In some embodiments, the controller (or surgeon via the controller) can reset or redefine the boundaries during a surgical procedure, e.g., as instruments are exchanged and/or a different part of the procedure is being performed.

At 502, the controller can determine a position of the end effector of the instrument. As described with reference to FIGS. 1 and 2, the instrument can be supported by a slave manipulator or robotic arm. In some embodiments, the controller can be configured to determine a position of the end effector based on kinematics and dimensions of the robotic arm supporting the instrument. Alternatively, or additionally, the controller can be configured to determine a position of the end effector using sensor data obtained from one or more sensors disposed on the robotic arm. Alternatively, or additionally, the controller can be configured to determine a position of the end effector using sensor data obtained from one or more sensors disposed on the instrument and/or end effector.

At 504, the controller can be configured to detect that the position of the end effector is within a predefined zone (e.g., zone 412) or predefined distance from a boundary of the workspace. In response to determining that the position of the end effector is within the predefined zone or distance, the controller can determine a percentage or factor to modify or adjust a commanded speed of the end effector, at 508. In some embodiments, the controller can determine a percentage or factor to modify or adjust a commanded speed of a translational movement of the end effector while maintaining a direction of the translational movement of the end effector. The controller can determine the percentage or factor to modify the commanded speed of the end effector based on a function of a distance of the end effector to the boundary, as described above with reference to FIGS. 2 and 4A. At 512, the controller can modify the commanded speed of the end effector by the percentage or factor.

The controller can continue monitoring the position of the end effector and adjusting the commanded speed of the end effector according to the method 500 during a surgical procedure. In other words, the method 500 can repeat or continue iterating through 502-512 during a surgical procedure.

Referring now to FIG. 8, the method 600 implements adjustments to a commanded speed of the end effector which further account for whether the end effector is moving toward or away from a boundary of the workspace. As depicted in FIG. 8, the method 600 can include optionally defining or setting one or more boundaries of a workspace, at 601, e.g., similar to 501 of method 500. The method 600 can include determining a current position of the end effector of the instrument, at 602, e.g., similar to 502 of method 500. The method 600 can include detecting that the position of the end effector is within a predefined zone or distance from a boundary of the workspace, at 604, e.g., similar to 504 of method 500.

At 606, the controller can determine whether the end effector is moving toward or away from the boundary. When the end effector is moving toward the boundary, then at 608, the controller can determine a percentage or factor to modify the commanded speed of the end effector based on a first function, e.g., a first function of the distance of the end effector to the boundary. When the end effector is moving away from the boundary, then at 610, the controller can determine a percentage or factor to modify the commanded speed of the end effector based on a second function, e.g., a second function of the distance of the end effector to the boundary. The commanded speed of the end effector can then be modified, at 612, similar to 512 of method 500. The controller can then repeat or continue iterating through 602-612 during a surgical procedure.

Referring to FIG. 9, the method 700 implements adjustments to the commanded speed of a slave manipulator and/or speed of an end effector of an instrument, which further accounts for one or more virtual fixtures. As depicted in FIG. 8, the method 700, at 701, can optionally include defining or setting one or more workspace boundaries, e.g., workspace boundaries WB as described with reference to FIG. 2. The workspace boundaries can define a workspace within which the instrument can move, e.g., as described with reference to FIGS. 3-5B. The method 700 can also optionally include defining or setting one or more virtual fixtures, e.g., virtual fixtures VF as described above with reference to FIG. 2. The virtual fixtures VF can limit the movement of the slave manipulator, e.g., to avoid collisions with physical objects in an environment around the surgical robotic system and/or to avoid certain zones or areas within the environment.

The method 700 can include receiving signals at a master controller indicative of a surgeon input (e.g., input 202), at 702. For example, the method 700 can include receiving commands by a surgeon at a master console (e.g., master console 110). As described above, the master console can include one or more master manipulators that can be manipulated by a surgeon to provide inputs for controlling one or more slave manipulators. The master manipulators can include one or more joint encoders that can output information regarding the position or angle of the joints of the master manipulators, which can be received at the controller. The controller can then determine a commanded speed vector for controlling the one or more slave manipulators based on the surgeon input or commands, at 704.

The method 700 can include determining a current position of a portion of the one or more slave manipulators (e.g., a hub or distal end of a slave manipulator), at 706. As described with reference to FIGS. 1 and 2, the position and movement of the slave manipulators can be controlled by the master manipulators, e.g., based on a speed vector. In some embodiments, the controller can be configured to determine the position of a portion of a slave manipulator based on sensors (e.g., encoders) disposed along the slave manipulator. Alternatively or additionally, the controller can be configured to determine the position of a portion of the slave manipulator based on the commanded speed vectors and a kinematic model. Other suitable mechanisms for determining the position of the portion of the slave manipulator can also be used, e.g., tracking of markers, cameras, etc. Optionally, the method 700 can also include determining a current position of the end effector of the instrument, at 706, e.g., similar to 502 of method 500.

At 710-712, the controller can be configured to determine whether to modify the commanded speed of the end effector and/or joints of the manipulator based on the determined positions of the portion of the manipulator and/or the end effector of the instrument, and to apply one or more function(s) to modify the commanded speed of the slave manipulator and/or end effector. In embodiments, the controller can be configured to modify the commanded speed vector for controlling the movement of the slave manipulator using a modulation gain, as described above with respect to FIGS. 2 and 6. For example, the controller can apply a modulation gain to the commanded speed vector based on a normal distance and the commanded normal speed of the robotic arm to a surface of a virtual fixture VF. Optionally, the controller can be configured to determine that the position of the end effector is within a predefined zone or predefined distance from a workspace boundary, and in response to determining such, modify or adjust the commanded speed of the translational movement of the end effector. This process can be similar to that described with respect to 504-512 of method 500 and 604-612 of method 600. These modifications can result in a modified speed vector for controlling the movement of a slave manipulator. The controller can then repeat or continue iterating through 702-712 during a surgical procedure.

FIG. 10 schematically illustrates the motion control of a robotic surgical system adapted for modulation or modification of the commanded speed of a slave manipulator and/or instrument coupled thereto, according to embodiments. As depicted in FIG. 10, inputs received at a master console 910 (e.g., structurally and/or functionally similar to master console 110) can be received by a controller (e.g., controller 114 and/or 124) implementing a motion control algorithm 950. The motion control 950 can include a commanded speed vector determination 952, in which the inputs received at the master console 910 (e.g., commands by a surgeon that result in encoder outputs associated with joints of one or more master manipulator(s)) can be converted into a nominal or commanded speed vector for controlling one or more slave manipulator(s) of a slave console 920 (e.g., structurally and/or functionally similar to slave console 120). The motion control 950 can also include speed modulation 954, which can apply one or more functions as described above to modulate or modify the commanded speed vector. The speed modulation 954 can determine whether and how to modulate or modify the commanded speed vector based on virtual fixture or boundary information 952 (e.g., similar to that described with reference to FIG. 2), the position of an end effector and/or portion of a slave manipulator (e.g., slave hub) received from the slave control 920, and the commanded speed vector. This process can be similar to the method 500, 600, and 700 described above with reference to FIGS. 7-9. The speed modulation 954 can then output a modulated or modified speed vector, which can be used to determine motor setpoints for controlling the movement of the slave manipulator(s) of the slave control 920.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

The indefinite articles “a” and “an,” as used herein in the specification and in the claims, unless clearly indicated to the contrary, should be understood to mean “at least one.”

1. A method of modulating a translational movement of an end effector of an instrument near a boundary of a virtual workspace, the method comprising:
determining that the end effector is within a predefined zone adjacent to the boundary; and
in response to determining that the end effector is within the predefined zone, controlling one or more actuators of an articulated instrument arm supporting the instrument to move the instrument such that a direction of the translational movement of the end effector is maintained while a commanded speed of the translational movement of the end effector is modified according to a function of a distance of the end effector to the boundary when the direction of the translational movement is toward the boundary, and according to a different function of the distance of the end effector to the boundary when the direction of the translational movement is away from the boundary.

2. The method of claim 1, wherein the translational movement of the end effector is modified by a percentage or factor that is determined by the function of the distance of the end effector to the boundary.

3.-7. (canceled)

8. The method of claim 2, wherein the percentage or factor as determined by the function decreases at a first rate as the distance of the end effector to the boundary decreases, and the percentage or factor as determined by the different function increases at a second rate as the distance of the end effector to the boundary increases, the second rate being greater than the first rate.

9. The method of claim 2, wherein the percentage or factor as determined by the function decreases at a first rate as the distance of the end effector to the boundary decreases, and the percentage or factor as determined by the different function increases at a second rate as the distance of the end effector to the boundary increases, the second rate being the same or substantially the same as the first rate.

10. The method of claim 1, wherein the predefined zone is defined based on one or more of a set distance away from the boundary and a set angle of the instrument relative to the boundary.

11. The method claim 1, wherein determining that the end effector is within the predefined zone includes determining, based on kinematics and dimensions of the articulated instrument arm and the instrument, a position of the end effector relative to the boundary.

12. The method of claim 1, wherein determining that the end effector is within the predefined zone includes determining, based on sensor data, a position of the end effector relative to the boundary.

13. The method of claim 12, further comprising:
obtaining the sensor data using one or more sensors disposed on the articulated instrument arm.

14. The method of claim 12, further comprising:
obtaining the sensor data using one or more sensors disposed on the instrument.

15. The method of claim 1, wherein the instrument is configured to operate within a body cavity, the method further comprising:
defining, before the instrument is inserted into the body cavity, the boundary based on one or more dimensions of the body cavity.

16.-24. (canceled)

25. An apparatus, comprising:
an articulated instrument arm supporting an instrument including an end effector, the articulated instrument arm including one or more actuators that are configured to drive a movement of the instrument within a virtual workspace defined by a boundary within a body cavity; and
a controller operatively coupled to the articulated instrument, the controller configured to:
monitor a position of the end effector within the body cavity;
determine, based on monitoring the position of the end effector, that the end effector is within a predefined zone adjacent to the boundary; and
in response to determining that the end effector is within the predefined zone, controlling the one or more actuators to move the instrument such that a direction of the translational movement of the end effector is maintained while a commanded speed of the translational movement of the end effector is modified according to a function of a distance of the end effector to the boundary when the direction of the translational movement is toward the boundary, and according to a different function of the distance of the end effector to the boundary when the direction of the translational movement is away from the boundary.

26. The apparatus of claim 25, wherein the controller is configured to control the one or more actuators to move the instrument such that the translational movement of the end effector is modified by a percentage or factor that is determined by the function of the distance of the end effector to the boundary.

27.-31. (canceled)

32. The apparatus of claim 26, wherein the percentage or factor as determined by the function decreases at a first rate as the distance of the end effector to the boundary decreases, and the percentage or factor as determined by the different function increases at a second rate as the distance of the end effector to the boundary increases, the second rate being greater than the first rate.

33. The apparatus of claim 25, wherein the percentage or factor as determined by the function decreases at a first rate as the distance of the end effector to the boundary decreases, and the percentage or factor as determined by the different function increases at a second rate as the distance of the end effector to the boundary increases, the second rate being the same or substantially the same as the first rate.

34. A method of modulating movement of an articulated instrument arm and an instrument coupled thereto, the method comprising:
determining a position of a distal portion of the articulated instrument arm relative to a virtual surface disposed outside of a body of a patient;
determining a position of an end effector of the instrument relative to a boundary of a virtual workspace disposed within the body of the patient;
adjusting a commanded speed vector of the articulated instrument arm based on at least on one of a distance between the position of the distal portion of the articulated instrument arm and the virtual surface or a distance between the position of the end effector and the boundary of the virtual workspace, to produce a modified speed vector; and
controlling one or more actuators of the articulated instrument arm to move the articulated instrument arm and the instrument based on the modified speed vector.

35. (canceled)

36. The method of claim 34, wherein the distance between the position of the distal portion of the articulated instrument arm and the virtual surface is a normal distance.

37. The method of claim 34, wherein adjusting the commanded speed vector of the articulated instrument arm includes:
determining a gain using a function of the commanded speed vector and the distance between the position of the distal portion of the articulated instrument arm and the virtual surface; and
applying the gain to the commanded speed vector to produce the modified speed vector.

38.-40. (canceled)

41. The method of claim 34, wherein adjusting the commanded speed vector of the articulated instrument arm includes:
determining a percentage or factor using a function of the distance between the position of the end effector and the boundary of the virtual workspace;
determining a modification to a commanded speed of translational movement of the end effector based on the percentage or factor; and
adjusting the commanded speed vector to implement the modification to the commanded speed of the translational movement of the end effector.

42.-43. (canceled)

44. The method of claim 34, wherein adjusting the commanded speed vector of the articulated instrument arm includes:
determining a percentage or factor using a first function of the distance between the position of the end effector and the boundary of the virtual workspace when a direction of translational movement of the end effector is toward the boundary and using a second function of the distance between the position of the end effector and the boundary when the direction of the translational movement is away from the boundary;
determining a modification to a commanded speed of a translational movement of the end effector based on the percentage or factor; and
adjusting the commanded speed vector to implement the modification to the commanded speed of the translational movement of the end effector.

45. (canceled)

46. The method of claim 44, wherein the percentage or factor as determined by the first function decreases at a first rate as the distance between the position of the end effector and the boundary decreases, and the percentage or factor as determined by the second function increases at a second rate as the distance between the position of the end effector and the boundary increases, the second rate being greater than the first rate.

47.-93. (canceled)
